(19) United States
(12) Patent Application Publication
(10) Pub. No.: US 2010/0226959 A1
(43) Pub. Date: Sep. 9, 2010

(54) MATRIX THAT PROLONGS GROWTH FACTOR RELEASE

(75) Inventor: William F. Mckay, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/397,405

(22) Filed: Mar. 4, 2009

(51) Int. Cl. A61L 27/54

(52) U.S. Cl. 424/425

(57) ABSTRACT

An implantable matrix is provided, the matrix having a porous interior configured to release a growth factor and to allow influx of at least progenitor, bone and/or cartilage cells therein; and a biodegradable membrane disposed on the porous interior, the biodegradable membrane being less porous than the porous interior and configured to retain the growth factor and release the growth factor from the porous interior as the biodegradable membrane degrades at or near the target tissue site. In some embodiments, a method for making the implantable collagen matrix is provided, the method comprising: providing a porous collagen layer configured to release a growth factor and to allow influx of at least progenitor, bone and/or cartilage cells therein, and disposing a collagen membrane on the porous collagen layer, the collagen membrane being less porous than the porous collagen layer and configured to retain the growth factor.

### BACKGROUND

Bone is a composite material that is composed of impure hydroxyapatite, collagen and a variety of non-collagenous proteins, as well as embedded and adherent cells. Due to disease, a congenital defect or an accident, a person may lose or be missing part or all of one or more bones or regions of cartilage in his or her body, and/or have improper growth or formation of bone and/or cartilage.

Mammalian bone tissue is known to contain one or more proteinaceous materials that are active during growth and natural bone healing. These materials can induce a developmental cascade of cellular events that results in bone formation. Typically, the developmental cascade of bone formation involves chemotaxis of mesenchymal cells, proliferation of progenitor cells, differentiation of cartilage, vascular invasion, bone formation, remodeling and marrow differentiation.

When bone is damaged, often bone grafting procedures are performed to repair the damaged bone especially in cases where the damage is complex, poses a significant risk to the patient, and/or fails to heal properly. Bone grafting is also used to help fusion between vertebrae, correct deformities, or provide structural support for fractures of the spine. In addition to fracture repair, bone grafting is also used to repair defects in bone caused by birth defects, traumatic injury, or surgery for bone cancer.

There are at least three ways in which a bone graft can help repair a defect. The first is called osteogenesis, the formation of new bone within the graft. The second is osteoinduction, a process in which molecules contained within the graft (e.g., bone morphogenic proteins) convert the patient's cells into cells that are capable of forming bone. The third is osteoconduction, a physical effect by which a matrix often containing graft material acts as a scaffold on which bone and cells in the recipient are able to form new bone.

The source of bone for grafting can be obtained from bones in the patient's own body (e.g., hip, skull, ribs, etc.), called autograft, or from bone taken from other people that is frozen and stored in tissue banks, called allograft. The source of bone may also be derived from animals of a different species called a xenograft.

Some grafting procedures utilize a variety of natural and synthetic matrices with or instead of bone (e.g., collagen, silicone, acrylics, hydroxyapatite, calcium sulfate, ceramics, etc.). To place the matrix at the bone defect, the surgeon makes an incision in the skin over the bone defect and shapes the matrix to fit into the defect. As persons of ordinary skill are aware, growth factors (e.g., bone morphogenic protein-2) may be placed on the matrix in order to spur the patient's body to begin the formation of new bone and/or cartilage. These growth factors act much like a catalyst, encouraging the necessary cells (including, but not limited to, mesenchymal stem cells, osteoblasts, and osteoclasts) to more rapidly migrate into the matrix, which is eventually resorbed via a cell-mediated process and newly formed bone is deposited at or near the bone defect. In this manner severe fractures may be healed, and vertebrae successfully fused.

Sometimes when the surgeon manipulates the matrix to place it in the bone defect, excessive amounts of growth factor (e.g., bone morphogenic protein) may leak from the matrix, which may reduce a stable microenvironment for new bone and/or cartilage growth. Other times blood and other bodily fluid will soak the matrix leading to excessive dilution and loss of the growth factor. It also may cause the matrix to fail to retain its full efficacy over time to maximally promote bone and/or cartilage growth at a target site. Thus, there is a need to develop new matrices that improve bone and/or cartilage repair.

### SUMMARY

Compositions and methods are provided that improve bone and/or cartilage repair. Through the use of matrices that prolong or slow growth factor release, the growth of bone, cartilage and/or related tissue may be facilitated. In some embodiments, the matrix provided serves to prolong residence time of the growth factor in or on the matrix by temporarily reducing the influx of blood and other bodily fluid into the matrix. This influx of blood and fluid, in some embodiments, can cause unwanted release of the growth factor. In this way, the matrix can maintain its efficacy over time to promote bone and/or cartilage growth at a target site.

In some embodiments, an implantable matrix configured to fit at or near a target tissue site is provided, the matrix comprising: a porous interior configured to release a growth factor and to allow influx of at least progenitor, bone and/or cartilage cells therein; and a biodegradable membrane disposed on the porous interior, the biodegradable membrane being less porous than the porous interior and configured to retain the growth factor and slow the release of the growth factor from the porous interior as the biodegradable membrane degrades at or near the target tissue site.

In some embodiments, the matrix of the present application can reduce or prevent compression of the matrix from occurring during or after implantation. Compression of the matrix causes the growth factor to be forced into surrounding environment, which may lead to unwanted adverse events such as local transient bone resorption. Compression of the matrix can also cause buffer from the bone growth factor to leak from the matrix, which causes a higher concentration of the growth factor (e.g., rhBMP-2) to remain on the matrix. This high concentration of growth factor may lead to unwanted adverse events such as local transient bone resorption as well. By disposing a membrane on or in the matrix, leakage of the growth factor is prevented or reduced.

In some embodiments, by using a membrane disposed on or in the porous interior, the growth factor (e.g., rhBMP-2) will be remain more evenly distributed throughout the interior of the matrix facilitating more uniform bone growth throughout the whole matrix. By allowing the growth factor to stay more evenly distributed within the interior of the matrix and thus avoids uneven distribution of the growth factor, for example, where a low dose of growth factor is distributed in the upper portion of the matrix, which may promote unwanted cartilage or soft tissue formation at the target tissue site.

In some embodiments, an implantable matrix configured to fit at or near a target tissue site is provided, the matrix comprising: a porous biodegradable interior configured to release a growth factor and to allow influx of at least progenitor and/or bone and/or cartilage cells therein; and a biodegradable membrane disposed on the porous biodegradable interior, the biodegradable membrane being less porous than the biodegradable interior and configured to retain the growth factor and slow the release of the growth factor from the porous biodegradable interior as the biodegradable membrane degrades at or near the target tissue site.

In some embodiments, a method for making an implantable collagen matrix is provided, the method comprising: providing a porous collagen layer configured to release a growth factor and to allow influx of at least progenitor and/or bone and/or cartilage cells therein, and disposing a collagen membrane on the porous collagen layer, the collagen membrane being less porous than the porous collagen layer and configured to retain the growth factor.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE FIGURES

In part, other aspects, features, benefits and advantages of the embodiments will be apparent with regard to the following description, appended claims and accompanying drawings where:

FIG. 1 is a magnified side sectional view of an embodiment of the implantable matrix shown as a bi-layered collagen sponge.

FIG. 1A illustrates a magnified view of the porous interior of the implantable matrix. The porous interior contains collagen and pores that are, in this view, open and function to release the growth factor and allow the influx of cells to aid in repair of bone and/or cartilage.

FIG. 1B illustrates a magnified view of the porous interior of the implantable matrix having pores that are closed, blocked, or clogged by a biodegradable membrane, which is less porous than the porous interior and configured to retain the growth factor. In this view, the membrane provided serves to prolong residence time of the growth factor in or on the matrix by temporarily limiting the presence of open channels for fluid exchange in and out of the matrix.

FIG. 2 is a magnified side sectional view of an embodiment of the implantable matrix being filled with the growth factor. In this illustrated embodiment, the matrix is shown as a bi-layered sponge.

FIG. 3 illustrates a side view of a vertebrae and the implantable matrix shaped as a plug with ridges for implantation at a target tissue site shown as an intervertebral site on the anterior side of the vertebral column.

FIG. 4 illustrates a number of common locations within a patient that may be sites at which the matrix can be implanted before, during or after surgery. It will be recognized that the locations illustrated in FIG. 4 are merely exemplary of the many different locations within a patient that the matrix can be implanted.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present application. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values are as precise as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Additionally, unless defined otherwise or apparent from context, all technical and scientific terms used herein have the same meanings as commonly understood by one of ordinary skill in the art to which this invention belongs.

Unless explicitly stated or apparent from context, the following terms or phrases have the definitions provided below:

Definitions

It is noted that, as used in this specification and the appended claims, the singular forms “a,” “an,” and “the,” include plural referents unless expressly and unequivocally limited to one referent. Thus, for example, reference to “a matrix” includes one, two, three or more matrices.

The term “biodegradable” includes that all or parts of the matrix will degrade over time by the action of enzymes, by hydrolytic action and/or by other similar mechanisms in the human body. In various embodiments, “biodegradable” includes that a matrix (e.g., sponge, sheet, etc.) can break down or degrade within the body to non-toxic components after or while a therapeutic agent has been or is being released. By “bioerodible” it is meant that the matrix will erode or degrade over time due, at least in part, to contact with substances found in the surrounding tissue, fluids or by cellular action. By “bioabsorbable” or “bioresorbable” it is meant that the matrix will be broken down and absorbed within the human body, for example, by a cell or tissue. “Biocompatible” means that the matrix will not cause substantial tissue irritation or necrosis at the target tissue site.

The term “mammal” refers to organisms from the taxonomy class “mammalian,” including but not limited to humans, other primates such as chimpanzees, apes, orangutans and monkeys, rats, mice, cats, dogs, cows, horses, etc.

The term “target tissue site” is intended to mean the location of the tissue to be treated. Typically the placement site of the matrix will be the same as the target site to provide for optimal targeted drug delivery. However, the present application also contemplates positioning the matrix at a placement site at or near the target site such that the therapeutic agent (e.g., growth factor) can be delivered to the surrounding vasculature, which carries the agent to the desired nearby target site. As used herein, the term “at or near” includes embodiments where the placement site and target site are within close proximity.

A “therapeutically effective amount” or “effective amount” is such that when administered, the drug (e.g., growth factor) results in alteration of the biological activity, such as, for example, promotion of bone, cartilage and/or other tissue (e.g., vascular tissue) growth, inhibition of inflammation, reduction or alleviation of pain, improvement in the condition through inhibition of an immunologic response, etc. The dosage administered to a patient can be as single or multiple doses depending upon a variety of factors, including the drug's administered pharmacokinetic properties, the route of administration, patient conditions and characteristics (sex, age, body weight, health, size, etc.), extent of symptoms, concurrent treatments, frequency of treatment and the effect desired. In some embodiments the implantable matrix is designed for sustained release. In some embodiments, the implantable matrix comprises an effective amount of a growth factor.

The phrase “immediate release” is used herein to refer to one or more therapeutic agent(s) that is introduced into the body and that is allowed to dissolve in or become absorbed at the location to which it is administered, with no intention of delaying or prolonging the dissolution or absorption of the drug.

The phrases “prolonged release”, “sustained release” or “sustain release” (also referred to as extended release or controlled release) are used herein to refer to one or more therapeutic agent(s) that is introduced into the body of a human or other mammal and continuously or continually releases a stream of one or more therapeutic agents over a predetermined time period and at a therapeutic level sufficient to achieve a desired therapeutic effect throughout the predetermined time period. Reference to a continuous or continual release stream is intended to encompass release that occurs as the result of biodegradation in vivo of the matrix and/or component thereof, or as the result of metabolic transformation or dissolution of the therapeutic agent(s) or conjugates of therapeutic agent(s). The release need not be linear and can be pulse type dosing.

The “matrix” of the present application is utilized as a scaffold for bone and/or cartilage repair, regeneration, and/or augmentation. Typically, the matrix provides a 3-D matrix of interconnecting pores, which acts as a pliant scaffold for cell migration. The morphology of the matrix guides cell migration and cells are able to migrate into or over the matrix, respectively. The cells then are able to proliferate and synthesize new tissue and form bone and/or cartilage.

The term “membrane” as used herein includes a sheet, strip, section or layer. Typically, the membrane will have no pores or pores having an average pore size of under 50 μm (e.g., 10 μm-50 μm), which is sufficiently small so as to prevent influx of cells and or bodily fluid (e.g., blood, CSF, etc.). The membrane is disposed on or in the porous interior of the matrix and prevents outward movement of the growth factor, as well as blood and other bodily fluid's influx into the matrix, which may cause an initial burst release of the growth factor from the matrix. When the membrane degrades, a gradual or prolonged or sustained release of the growth factor will occur as the membrane degrades.

The terms “treating” and “treatment” when used in connection with a disease or condition refer to executing a protocol that may include a repair procedure (e.g., osteochondral repair procedure), administering one or more matrices to a patient (human or other mammal), in an effort to alleviate signs or symptoms of the disease or condition or immunological response. Alleviation can occur prior to signs or symptoms of the disease or condition appearing, as well as after their appearance. Thus, treating or treatment includes preventing or prevention of disease or undesirable condition. In addition, treating, treatment, preventing or prevention do not require complete alleviation of signs or symptoms, does not require a cure, and specifically includes protocols that have only a marginal effect on the patient. In some embodiments, the implantable matrix can be used to treat subchondral, osteochondral, hyaline cartilage and/or condyle defects.

The term “subchondral” includes an area underlying joint cartilage. The term “subchondral bone” includes a very dense, but thin layer of bone just below a zone of cartilage and above the cancellous or trabecular bone that forms the bulk of the bone structure of the limb. “Osteochondral” includes a combined area of cartilage and bone where a lesion or lesions can occur. “Osteochondral defect” includes a lesion, which is a composite lesion of cartilage and subchondral bone. “Hyaline cartilage” includes cartilage containing groups of isogenous chondrocytes located within lacunae cavities which are scattered throughout an extracellular collagen matrix. A “condyle” includes a rounded articular surface of the extremity of a bone.

The matrix comprising the growth factor may be osteogenic. The term “osteogenic” as used herein includes the ability of the matrix to enhance or accelerate the growth of new bone tissue by one or more mechanisms such as osteogenesis, osteoconduction and or osteoinduction. In some embodiments, the matrix is osteogenic and can be delivered to other surgical sites, particularly sites at which bone growth is desired. These include, for instance, the repair of spine (e.g., vertebrae fusion) cranial defects, iliac crest back-filling, acetabular defects, in the repair of tibial plateau, long bone defects, spinal site defects or the like. Such methods can be used to treat major or minor defects in these or other bones caused by trauma (including open and closed fractures), disease, or congenital defects, for example.

The matrix comprising the growth factor may be osteoinductive. The term “osteoinductive” as used herein includes the ability of a substance to recruit cells from the host that have the potential for forming new bone and repairing bone tissue. Most osteoinductive materials can stimulate the formation of ectopic bone in soft tissue.

The matrix comprising the growth factor may be osteoconductive. The term “osteoconductive” as utilized herein includes the ability of a non-osteoinductive substance to serve as a suitable template or substrate along which bone may grow.

The matrix may be implantable. The term “implantable” as utilized herein refers to a biocompatible device retaining potential for successful placement within a mammal. The expression “implantable device” and expressions of like import as utilized herein refers to any object implantable through surgery, injection, or other suitable means whose primary function is achieved either through its physical presence or mechanical properties.

The term “carrier” includes a diluent, adjuvant, buffer, excipient, or vehicle with which a composition can be administered. Carriers can include sterile liquids, such as, for example, water and oils, including oils of petroleum, animal, vegetable or synthetic origin, such as, for example, peanut oil, soybean oil, mineral oil, sesame oil, or the like. The growth factor may include a carrier.

The term “excipient” includes a non-therapeutic agent added to a pharmaceutical composition to provide a desired consistency or stabilizing effect. Excipients for parenteral formulations, include, for example, oils (e.g., canola, cottonseed, peanut, safflower, sesame, soybean), fatty acids and salts and esters thereof (e.g., oleic acid, stearic acid, palmitic acid), alcohols (e.g., ethanol, benzyl alcohol), polyalcohols (e.g., glycerol, propylene glycols and polyethylene glycols, e.g., PEG 3350), polysorbates (e.g., polysorbate 20, polysorbate 80), gelatin, albumin (e.g., human serum albumin), salts (e.g., sodium chloride), succinic acid and salts thereof (e.g., sodium succinate), amino acids and salts thereof (e.g., alanine, histidine, glycine, arginine, lysine), acetic acid or a salt or ester thereof (e.g., sodium acetate, ammonium acetate), citric acid and salts thereof (e.g., sodium citrate), benzoic acid and salts thereof, phosphoric acid and salts thereof (e.g., monobasic sodium phosphate, dibasic sodium phosphate), lactic acid and salts thereof, polylactic acid, glutamic acid and salts thereof (e.g., sodium glutamate), calcium and salts thereof (e.g., CaCl₂, calcium acetate), phenol, sugars (e.g., glucose, sucrose, lactose, maltose, trehalose), erythritol, arabitol, isomalt, lactitol, maltitol, mannitol, sorbitol, xylitol, nonionic surfactants (e.g., TWEEN 20, TWEEN 80), ionic surfactants (e.g., sodium dodecyl sulfate), chlorobutanol, DMSO, sodium hydroxide, glycerin, m-cresol, imidazole, protamine, zinc and salts thereof (e.g, zinc sulfate), thimerosal, methylparaben, propylparaben, carboxymethylcellulose, chlorobutanol, or heparin. The growth factor may include an excipient.

The term “lyophilized” or “freeze-dried” includes a state of a substance that has been subjected to a drying procedure such as lyophilization, where at least 50% of moisture has been removed. The matrix and/or growth factor may be lyophilized or freeze-dried.

A “preservative” includes a bacteriostatic, bacteriocidal, fungistatic or fungicidal compound that is generally added to formulations to retard or eliminate growth of bacteria or other contaminating microorganisms in the formulations. Preservatives include, for example, benzyl alcohol, phenol, benzalkonium chloride, m-cresol, thimerosol, chlorobutanol, methylparaben, propylparaben and the like. Other examples of pharmaceutically acceptable preservatives can be found in the USP. The growth factor and/or matrix may have preservatives or be preservative free.

Reference will now be made in detail to certain embodiments of the invention. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

Compositions and methods are provided that improve bone and/or cartilage repair. Through the use of matrices that prolong or slow growth factor release, the growth of bone, cartilage and/or related tissue may be facilitated.

In some embodiments, by employing a membrane on or in the interior matrix, loss of the growth factor and/or buffer is prevented and/or reduced during implantation of the matrix. The matrix will then degrade after a couple of days after it is implanted to allow progenitor cell infiltration. The progenitor cells migrate into the matrix within a few days of implantation to ensure good bone growth within the matrix and then at the target tissue site.

In some embodiments, the matrix provided serves to prolong residence time of the growth factor in or on the matrix by temporarily limiting the presence of open channels for fluid exchange in and out of the matrix. In this way, the matrix can maintain its efficacy over time to promote bone and/or cartilage growth at a target site. In addition, it has been found that the influx of blood and fluid, in some embodiments, can cause unwanted release of the growth factor by de-binding it from the matrix. By employing a membrane on or in the interior matrix, leakage of the growth factor is prevented and/or reduced.

In some embodiments, the matrix of the present application can reduce or prevent compression of the implantable matrix from occurring. Often compression of the implantable matrix causes the growth factor to be forced into surrounding environment, which may lead to unwanted adverse events such as local transient bone resorption. Compression of the matrix can also cause buffer from the bone growth factor to leak from the matrix, which causes a higher concentration of the growth factor (e.g., rhBMP-2) to remain on the matrix. This high concentration of growth factor may lead to local transient bone resorption as well. By employing a membrane on or in the interior matrix, leakage of the growth factor and/or buffer is prevented and/or reduced.

In some embodiments, the growth factor (e.g., rhBMP-2) will be more evenly distributed throughout the interior of the matrix and facilitate more uniform bone growth throughout the whole matrix. In some embodiments, the growth factor (e.g., rhBMP-2) is temporarily retained within the matrix so as to limit new bone formation to within the matrix.

By reducing compression, the matrix of the present application allows the growth factor to stay evenly distributed within the interior of the matrix and thus avoids uneven distribution of the growth factor, for example, where a low dose of growth factor is distributed in the upper portion of the matrix, which may promote cartilage or soft tissue formation at the target tissue site.

The headings below are not meant to limit the disclosure in any way; embodiments under any one heading may be used in conjunction with embodiments under any other heading.

### Matrix

The matrix provides a tissue scaffold for the cells to guide the process of tissue formation in vivo in three dimensions. The morphology of the matrix guides cell migration and cells are able to migrate into or over the matrix. The cells then are able to proliferate and synthesize new tissue and form bone and/or cartilage. In some embodiments, one or more tissue matrices are stacked on one another.

The matrix comprises a porous interior configured to release a growth factor and to allow influx of at least bone and/or cartilage cells therein; and a biodegradable membrane disposed on the porous interior, the biodegradable membrane being less porous than the porous interior and configured to retain the growth factor and release the growth factor from the porous interior as the biodegradable membrane degrades at or near the target tissue site. By porous is meant that the interior has a plurality of pores. The pores of the interior of the matrix are a size large enough to allow influx of blood, other bodily fluid, and progenitor and/or bone and/or cartilage cells into the interior to guide the process of tissue formation in vivo in three dimensions.

In some embodiments, the interior of the matrix comprises a plurality of pores. In some embodiments, at least 10% of the pores are between about 50 micrometers and about 500 micrometers at their widest points. In some embodiments, at least 20% of the pores are between about 50 micrometers and about 250 micrometers at their widest points. In some embodiments, at least 30% of the pores are between about 50 micrometers and about 150 micrometers at their widest points. In some embodiments, at least 50% of the pores are between about 10 micrometers and about 500 micrometers at their widest points. In some embodiments, at least 90% of the pores are between about 50 micrometers and about 250 micrometers at their widest points. In some embodiments, at least 95% of the pores are between about 50 micrometers and about 150 micrometers at their widest points. In some embodiments, 100% of the pores are between about 10 micrometers and about 500 micrometers at their widest points.

In some embodiments, the porous interior of the matrix has a porosity of at least about 30%, at least about 50%, at least about 60%, at least about 70%, at least about 90% or at least about 95%, or at least about 99%. The pores may support ingrowth of cells, formation or remodeling of bone, cartilage and/or vascular tissue.

The porous interior also holds the growth factor within the matrix and because the interior is porous, the growth factor is evenly distributed throughout the matrix when growth factor is injected into the matrix.

In some embodiments, growth factor will be held within the interior of the matrix and released into the environment surrounding the matrix (e.g., bone defect, osteochondral defect, etc.) as the membrane degrades over time. In some embodiments, the membrane is configured to degrade overtime (e.g., 1, 2, 3, 4, 5, 6 hours or longer) to allow progenitor cells within the matrix. The progenitor cells have a capacity to differentiate into a specific cell type with the ability to generate cartilage, bone, muscle, tendon, and/or ligaments in the matrix. For example, the matrix membrane can start to degrade within a few hours after implantation. This will allow the growth factor to remain within the matrix during the implantation process. As the membrane degrades (in some embodiments within a few hours), the pores of the matrix become more open and they allow progenitor cells in the matrix to begin to start forming bone, cartilage and/or other tissue within the matrix. In some embodiments, the membrane will completely degrade over 3 to 7 days after it is implanted. In this way, the matrix provided serves to prolong residence time of the growth factor in or on the matrix by temporarily limiting the presence of open channels for blood and/or fluid exchange in and out of the matrix. This influx of blood and fluid, in some embodiments, can cause unwanted release of the growth factor. In this way, the matrix containing the membrane can maintain the efficacy of the growth factor over time to promote bone and/or cartilage growth at a target site.

In some embodiments, the membrane allows release of the growth factor as cells migrate into the matrix, and the release of the growth factor can be, for example, from about 2 weeks to about 6 weeks or from about 3 weeks to 4 weeks.

The matrix comprises a membrane disposed on or in the porous interior. The membrane comprises one or more thin sheet(s), strip(s), region(s) or layer(s) disposed on or in the porous interior. The membrane is less porous than the porous interior and acts as a skin or shell around the porous interior. The thickness of this membrane is about 0.1-1.0 mm thick or about 0.5 mm to about 1 mm thick. Typically, the membrane will initially have no pores or less than 1-30% of its surface area to prevent or limit the efflux of fluid out of the matrix or less than 15% of its surface area to prevent or limit the efflux of fluid out of the matrix. Initial average pore size is often under 50 μm (e.g., 10 μm-100 μm), which is sufficiently small so as to prevent influx of cells and or bodily fluid (e.g., blood, CSF, etc.). The membrane is disposed on or in the porous interior of the matrix and prevents outward movement of the growth factor, as well as blood and other bodily fluid's influx into the matrix, which may cause an initial burst release of the growth factor from the matrix. When the membrane degrades over time, a gradual or prolonged or sustained release of the growth factor will occur.

By utilizing the membrane on the porous interior of the matrix, in some embodiments, the density of the matrix is increased when compared to a typical growth factor matrix (e.g., BMP sponge carrier) so that the release of the growth factor (e.g., BMP) is much slower. As such, the longer release kinetic properties of the matrix avoids the potential for local transient bone resorption, and instead a more rapid increase in bone deposition, which therefore ultimately achieves a higher bone mineral density. This is particularly beneficial in osteoporotic bone.

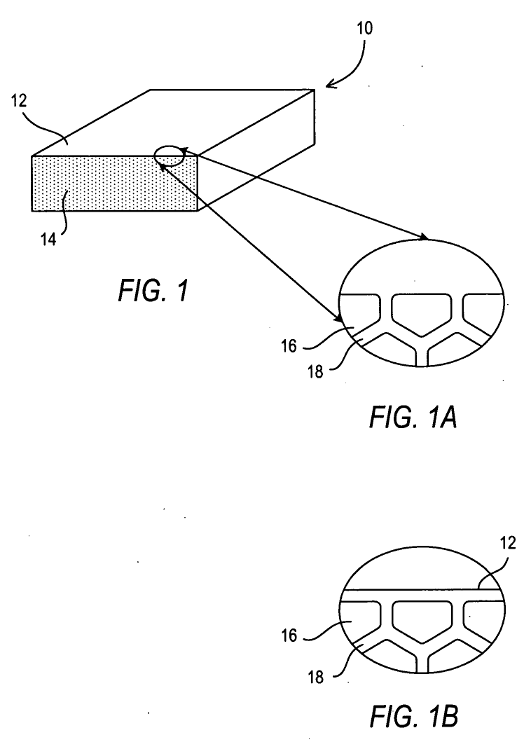

FIG. 1 is a magnified side sectional view of an embodiment of the matrix 10 shown as a bi-layered collagen sponge. In this illustrated embodiment, the matrix comprises a porous interior 14 configured to release a growth factor and to allow influx of at least bone and/or cartilage cells therein. The matrix comprises a biodegradable membrane 12 disposed on the porous interior, the biodegradable membrane being less porous than the porous interior and configured to retain the growth factor and release the growth factor from the porous interior as the biodegradable membrane degrades at or near the target tissue site. In FIG. 1A, the porous interior is shown magnified. The porous interior contains collagen 16 and pores 18 that are open and function to retain the growth factor and release the growth factor when the pores are open as shown in 18. The growth factor will be released to the environment surrounding the sponge and allow the growth factor to cause bone and/or cartilage growth. The porous interior of the sponge will also allow cell migration into or over the porous interior of the matrix to enhance bone and/or cartilage remodeling.

FIG. 1B shows a magnified view of the porous interior. The porous interior contains collagen 16 and pores 18. Illustrated in this embodiment, are pores that are closed, blocked, or clogged by a biodegradable membrane 12 disposed on the porous interior and being less porous than the porous interior. The biodegradable membrane 12 is configured to retain the growth factor. In this way, the matrix provided serves to prolong residence time of the growth factor in or on the matrix by temporarily limiting the presence of open channels for blood and other bodily fluid exchange in and out of the matrix. This influx of blood and fluid, in some embodiments, can cause unwanted release of the growth factor. Thus, the matrix can maintain the efficacy of the growth factor over time to promote bone and/or cartilage growth at a target site.

For example, in some embodiments, the collagen sponge can be coated by disposing a poly(L-lactide) (PLLA) and sucrose acetate isobutyrate-based (SAIB-based) coat around the sponge. The sponge can be loaded with the growth factor (e.g., rhBMP-2) and the release of the growth factor from the sponge will be prolonged (e.g., from 24 hours without the coat to 12 days with the coating).

In some embodiments, the membrane comprises biodegradable material. For example, the membrane may comprises one or more poly(alpha-hydroxy acids), poly(lactide-co-glycolide) (PLGA), polylactide (PLA), poly(L-lactide), polyglycolide (PG), polyethylene glycol (PEG) conjugates of poly(alpha-hydroxy acids), polyorthoesters (POE), polyaspirins, polyphosphagenes, collagen, hydrolyzed collagen, gelatin, hydrolyzed gelatin, fractions of hydrolyzed gelatin, elastin, starch, pre-gelatinized starch, hyaluronic acid, chitosan, alginate, albumin, fibrin, vitamin E analogs, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, POE, SAIB (sucrose acetate isobutyrate), polydioxanone, methylmethacrylate (MMA), MMA and N-vinylpyrrolidone, polyamide, oxycellulose, copolymer of glycolic acid and trimethylene carbonate, polyesteramides, polyetheretherketone, polymethylmethacrylate, silicone, hyaluronic acid, chitosan, or combinations thereof.

In some embodiments, the membrane has a thickness of from about 1 μm to about 1000 μm thick, or from about 50 μm to about 800 μm thick, or from about 100 μm to about 700 μm thick, or from about 200 μm to about 600 μm thick, or from about 300 μm to about 500 μm thick. In some embodiments, the membrane has a density of about 100 mg/cc to about 250 mg/cc, or 150 mg/cc to about 200 mg/cc.

In some embodiments, at least 10% of the pores of the membrane are between about 50 micrometers and about 500 micrometers at their widest points. In some embodiments, at least 20% of the pores of the membrane are between about 50 micrometers and about 250 micrometers at their widest points.

In some embodiments, the membrane does not have any pores. In other embodiments, at least 10% of the pores of the membrane are between about 10 micrometers and about 50 micrometers at their widest points. In some embodiments, at least 25% of the pores of the membrane are between about 10 micrometers and about 50 micrometers at their widest points. In some embodiments, at least 50% of the pores of the membrane are between about 10 micrometers and about 50 micrometers at their widest points. In some embodiments, at least 90% of the pores are between about 10 micrometers and about 50 micrometers at their widest points. In some embodiments, at least 95% of the pores are between about 10 micrometers and about 50 micrometers at their widest points. In some embodiments, 100% of the pores are between about 10 micrometers and about 50 micrometers at their widest points.

By disposing a membrane on or in the porous interior, when excessive compression occurs on the matrix, growth factor leakage from the matrix will be eliminated or reduced and the growth factor will have improved residence time in the matrix as well as uniform distributed in the matrix.

It will be understood by those of ordinary skill in the art that in some embodiments, some compression may occur to release the growth factor (e.g., less than 0.75 M Pa, or 0.5 M Pa, or 0.25 M Pa of pressure), which may release less than 5%, 4%, 3%, 2%, 1%, or 0.5% w/w or w/v of the growth factor. But the release will be minimal or non-existent because the membrane is disposed in or around the matrix.

The matrix including the interior may comprise natural and/or synthetic material. For example, the matrix may comprise poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters (POE), polyaspirins, polyphosphagenes, collagen, hydrolyzed collagen, gelatin, hydrolyzed gelatin, fractions of hydrolyzed gelatin, elastin, starch, pre-gelatinized starch, hyaluronic acid, chitosan, alginate, albumin, fibrin, vitamin E analogs, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate), POE, polydioxanone, methylmethacrylate (MMA), MMA and N-vinylpyrrolidone, polyamide, oxycellulose, copolymer of glycolic acid and trimethylene carbonate, polyesteramides, polyetheretherketone, polymethylmethacrylate, silicone, hyaluronic acid, chitosan, or combinations thereof.

The porous interior and the membrane of the matrix may be made from the same material or the porous interior may be made from different material from the membrane. In some embodiments, the entire matrix (including the membrane and interior) is biodegradable. In some embodiments, only the membrane is biodegradable.

In some embodiments, the matrix (including the porous interior and/or membrane) may comprise a resorbable ceramic (e.g., hydroxyapatite, tricalcium phosphate, bioglasses, calcium sulfate, etc.) tyrosine-derived polycarbonate poly (DTE-co-DT carbonate), in which the pendant group via the tyrosine—an amino acid—is either an ethyl ester (DTE) or free carboxylate (DT) or combinations thereof.

In some embodiments, the matrix (e.g., membrane and/or interior) comprises collagen. Exemplary collagens include human or non-human (bovine, ovine, and/or porcine), as well as recombinant collagen or combinations thereof. Examples of suitable collagen include, but are not limited to, human collagen type I, human collagen type II, human collagen type III, human collagen type IV, human collagen type V, human collagen type VI, human collagen type VII, human collagen type VIII, human collagen type IX, human collagen type X, human collagen type XI, human collagen type XII, human collagen type XIII, human collagen type XIV, human collagen type XV, human collagen type XVI, human collagen type XVII, human collagen type XVIII, human collagen type XIX, human collagen type XXI, human collagen type XXII, human collagen type XXIII, human collagen type XXIV, human collagen type XXV, human collagen type XXVI, human collagen type XXVII, and human collagen type XXVIII, or combinations thereof. Collagen further may comprise hetero- and homo-trimers of any of the above-recited collagen types. In some embodiments, the collagen comprises hetero- or homo-trimers of human collagen type I, human collagen type II, human collagen type III, or combinations thereof.

In some embodiments, the matrix comprises collagen-containing biomaterials from the implant market which, when placed in a bone defect, provide scaffolding around which the patient's new bone and/or cartilage will grow, gradually replacing the carrier matrix as the target site heals. Examples of suitable carrier matrices may include, but are not limited to, the MasterGraft® Matrix produced by Medtronic Sofamor Danek, Inc., Memphis, Tenn.; MasterGraft® Putty produced by Medtronic Sofamor Danek, Inc., Memphis, Tenn.; Absorbable Collagen Sponge (“ACS”) produced by Integra LifeSciences Corporation, Plainsboro, N.J.; bovine skin collagen fibers coated with hydroxyapatite, e.g. Healos®. marketed by Johnson & Johnson, USA; collagen sponges, e.g. Hemostagene® marketed by Coletica S A, France, or e.g. Helisat® marketed by Integra Life Sciences Inc., USA; and Collagraft® Bone Graft Matrix produced by Zimmer Holdings, Inc., Warsaw, Ind.

In some embodiments, the matrix may comprise particles of bone-derived materials. The bone-derived material may include one or more of non-demineralized bone particles, demineralized bone particles, lightly demineralized bone particles, and/or deorganified bone particles.

In some embodiments, the matrix may be seeded with harvested bone cells and/or bone tissue, such as for example, cortical bone, autogenous bone, allogenic bones and/or xenogenic bone. In some embodiments, the matrix may be seeded with harvested cartilage cells and/or cartilage tissue (e.g., autogenous, allogenic, and/or xenogenic cartilage tissue). For example, before insertion into the target tissue site, the matrix can be wetted with the graft bone tissue/cells, usually with bone tissue/cells aspirated from the patient, at a ratio of about 3:1, 2:1, 1:1, 1:3 or 1:2 by volume. The bone tissue/cells are permitted to soak into the matrix provided, and the matrix may be kneaded by hand or machine, thereby obtaining a pliable consistency that may subsequently be packed into the bone defect. In some embodiments, the matrix provides a malleable, non-water soluble carrier that permits accurate placement and retention at the implantation site. In some embodiments, the harvested bone and/or cartilage cells can be mixed with the growth factor and seeded in the interior of the matrix.

In some embodiments, the matrix may contain an inorganic material, such as an inorganic ceramic and/or bone substitute material. Exemplary inorganic materials or bone substitute materials include but are not limited to aragonite, dahlite, calcite, amorphous calcium carbonate, vaterite, weddellite, whewellite, struvite, urate, ferrihydrate, francolite, monohydrocalcite, magnetite, goethite, dentin, calcium carbonate, calcium sulfate, calcium phosphosilicate, sodium phosphate, calcium aluminate, calcium phosphate, hydroxyapatite, alpha-tricalcium phosphate, dicalcium phosphate, β-tricalcium phosphate, tetracalcium phosphate, amorphous calcium phosphate, octacalcium phosphate, BIOGLASS™, fluoroapatite, chlorapatite, magnesium-substituted tricalcium phosphate, carbonate hydroxyapatite, substituted forms of hydroxyapatite (e.g., hydroxyapatite derived from bone may be substituted with other ions such as fluoride, chloride, magnesium sodium, potassium, etc.), or combinations or derivatives thereof.

In some embodiments, by including inorganic ceramics, such as for example, calcium phosphate, in the matrix, this will facilitate the prevention of local bone resorption by providing slower release of the growth factor due to its increased binding potential and also act as a local source of calcium and phosphate to the cells attempting to deposit new bone.

In some embodiments, tissue will infiltrate the matrix to a degree of about at least 50 percent within about 1 month to about 6 months after implantation of the matrix. In some embodiments, about 75 percent of the matrix will be infiltrated by tissue within about 2-3 months after implantation of the matrix. In some embodiments, the matrix will be substantially, e.g., about 90 percent or more, submerged in or enveloped by tissue within about 6 months after implantation of the matrix. In some embodiments, the matrix will be completely submerged in or enveloped by tissue within about 9-12 months after implantation.

In some embodiments, the matrix has a thickness of from 1 mm to 15 mm, or from about 2 mm to about 10 mm, or 3 mm to about 5 mm. Clearly, different bone defects (e.g., osteochondral defects) may require different matrices thicknesses.

In some embodiments, the matrix has a density of between about 1.6 g/cm³, and about 0.05 g/cm³. In some embodiments, the matrix has a density of between about 1.1 g/cm³, and about 0.07 g/cm³. For example, the density may be less than about 1 g/cm³, less than about 0.7 g/cm³, less than about 0.6 g/cm³, less than about 0.5 g/cm³, less than about 0.4 g/cm³, less than about 0.3 g/cm³, less than about 0.2 g/cm³, or less than about 0.1 g/cm³.

The shape of the matrix may be tailored to the site at which it is to be situated. For example, it may be in the shape of a morsel, a plug, a pin, a peg, a cylinder, a block, a wedge, a sheet, a strip, etc. The term “shape” refers to a determined or regular form or configuration in contrast to an indeterminate or vague form or configuration (as in the case of a lump or other solid mass of no special form) and is characteristic of such materials as sheets, plates, disks, cores, tubes, wedges, cylinders, or the like. This includes forms ranging from regular, geometric shapes to irregular, angled, or non-geometric shapes, or combinations of features having any of these characteristics.

In some embodiments, the diameter or diagonal of the matrix can range from 1 mm to 50 mm. In some embodiments, the diameter or diagonal of the matrix can range from 1 mm to 30 mm, or 5 mm to 10 mm which is small enough to fit through an endoscopic cannula, but large enough to minimize the number of matrices needed to fill a large the bone defect (e.g., osteochondral defect).

### Method of Making Matrix

In some embodiments, the matrix may be made by injection molding, compression molding, blow molding, thermoforming, die pressing, slip casting, electrochemical machining, laser cutting, water-jet machining, electrophoretic deposition, powder injection molding, sand casting, shell mold casting, lost tissue scaffold casting, plaster-mold casting, ceramic-mold casting, investment casting, vacuum casting, permanent-mold casting, slush casting, pressure casting, die casting, centrifugal casting, squeeze casting, rolling, forging, swaging, extrusion, shearing, spinning, powder metallurgy compaction or combinations thereof.

One form of manufacturing the matrix involves casting the matrix material in a mold. The matrix material can take on the shape of the mold such as, crescent, quadrilateral, rectangular, cylindrical, plug, or any other shape. Additionally, the surface of the mold may be smooth or may include raised features or indentations to impart features to the matrix. Features from the mold can be imparted to the matrix as the matrix material in the mold is dried. In particular aspects, a roughened or friction engaging surface can be formed on the superior surface and/or the inferior surface of the matrix body. In some embodiments, protuberances or raised portions can be imparted on the superior surface and/or the inferior surface from the mold. Such examples of protuberances or raised portions are ridges, serrations, pyramids, and teeth, or the like.

In some embodiments, in manufacturing the matrix, a mixture of the matrix material (e.g., collagen) is combined with a liquid to wet the material and form a slurry. Any suitable liquid can be used including, for example, aqueous preparations such as water, saline solution (e.g. physiological saline), sugar solutions, protic organic solvents, or liquid polyhydroxy compounds such as glycerol and glycerol esters, or mixtures thereof. The liquid may, for example, constitute about 5 to about 70 weight percent of the mixed composition prior to the molding operation. Certain liquids such as water can be removed in part or essentially completely from the formed matrix using conventional drying techniques such as air drying, heated drying, lyophilization, or the like.

In one embodiment of manufacture, a collagen mixture can be combined with a liquid, desirably with an aqueous preparation, to form a slurry. Excess liquid can be removed from the slurry by any suitable means, including for example by applying the slurry to a liquid-permeable mold or form and draining away excess liquid.

Before, during or after molding, including in some instances the application of compressive force to the collagen containing material, the collagen material can be subjected to one or more additional operations such as heating, lyophilizing and/or crosslinking to make the porous collagen interior of the matrix and/or the membrane of the desired porosity. In this regard, crosslinking can be used to improve the strength of the formed matrix (membrane and/or porous interior). Alternatively, one or more of the surface of the matrix can be crosslinked to reduce the size of the pores of the porous interior and thereby form the membrane of the matrix that is less permeable and/or less porous than the porous interior. Crosslinking can be achieved, for example, by chemical reaction, the application of energy such as radiant energy (e.g. UV light or microwave energy), drying and/or heating and dye-mediated photo-oxidation; dehydrothermal treatment; enzymatic treatment or others.

Chemical crosslinking agents will generally be preferred, including those that contain bifunctional or multifunctional reactive groups, and which react with matrix. Chemical crosslinking can be introduced by exposing the matrix material to a chemical crosslinking agent, either by contacting it with a solution of the chemical crosslinking agent or by exposure to the vapors of the chemical crosslinking agent. This contacting or exposure can occur before, during or after a molding operation. In any event, the resulting material can then be washed to remove substantially all remaining amounts of the chemical crosslinker if needed or desired for the performance or acceptability of the final implantable matrix.

Suitable chemical crosslinking agents include mono- and dialdehydes, including glutaraldehyde and formaldehyde; polyepoxy compounds such as glycerol polyglycidyl ethers, polyethylene glycol diglycidyl ethers and other polyepoxy and diepoxy glycidyl ethers; tanning agents including polyvalent metallic oxides such as titanium dioxide, chromium dioxide, aluminum dioxide, zirconium salt, as well as organic tannins and other phenolic oxides derived from plants; chemicals for esterification or carboxyl groups followed by reaction with hydrazide to form activated acyl azide functionalities in the collagen; dicyclohexyl carbodiimide and its derivatives as well as other heterobifunctional crosslinking agents; hexamethylene diisocyante; and/or sugars, including glucose, will also crosslink the matrix material.

In some embodiments, to dispose the membrane on the porous interior of the matrix, the porous interior is optionally subjected to a process, which occludes, clogs, or blocks the pores or reduces the size of the pores to make it less permeable to tissue and/or cell ingrowth. This process can be performed by heating all or a portion of the surface area of the porous interior, by cross-linking all or a portion of the surface area of the porous interior, and/or by applying one or more biocompatible substances (e.g., collagen) to all or a portion of the surface area of the porous interior to provide a microporous layer or membrane thereon. It will be understood by those skilled in the art that combinations of one or more of the foregoing processes can be employed, e.g., heating followed by cross-linking, cross-linking followed by heating, cross-linking followed by application of biocompatible substance, etc. It will also be understood by those skilled in is the art that any of these processes can be performed on the porous interior before or after the optional drying and/or lyophilizing steps described above. Thus, e.g., the porous interior of the matrix can be subjected to the heating operation to occlude a portion or all of the surface area of the porous interior followed by lyophilization of the occluded surfaces of the porous interior.

In some embodiments, the porous interior of the matrix is formed and the membrane may be disposed on or in the interior of the matrix by hand, electrospraying, ionization spraying or impregnating, vibratory dispersion (including sonication), nozzle spraying, compressed-air-assisted spraying, dipping, brushing and/or pouring.

For example, in some embodiments, when the interior matrix comprises a collagen sponge, the pores of the collagen sponge can be spray coated or dipped with collagen to form the membrane. The pores of the interior matrix will now be blocked by the membrane and upon introduction of the growth factor in the interior of the matrix, the growth factor will have a prolonged residence time and sustain release from the sponge. The membrane will also prevent the influx of blood, fluid and/or cells into the matrix until it degrades.

In some embodiments, the matrix may comprise sterile and/or preservative free material. The matrix can be implanted by hand or machine in procedures such as for example, laparoscopic, arthroscopic, neuroendoscopic, endoscopic, rectoscopic procedures or the like.

The matrix of the present application may be used to repair bone and/or cartilage at a target tissue site, e.g., one resulting from injury, defect brought about during the course of surgery, infection, malignancy or developmental malformation. The matrix can be utilized in a wide variety of orthopedic, periodontal, neurosurgical, oral and maxillofacial surgical procedures such as the repair of simple and/or compound fractures and/or non-unions; external and/or internal fixations; joint reconstructions such as arthrodesis; general arthroplasty; cup arthroplasty of the hip; femoral and humeral head replacement; femoral head surface replacement and/or total joint replacement; repairs of the vertebral column including spinal fusion and internal fixation; tumor surgery, e.g., deficit filling; discectomy; laminectomy; excision of spinal cord tumors; anterior cervical and thoracic operations; repairs of spinal injuries; scoliosis, lordosis and kyphosis treatments; intermaxillary fixation of fractures; mentoplasty; temporomandibular joint replacement; alveolar ridge augmentation and reconstruction; inlay implantable matrices; implant placement and revision; sinus lifts; cosmetic procedures; etc. Specific bones which can be repaired or replaced with the implantable matrix herein include the ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, ilium, ischium, pubis, femur, tibia, fibula, patella, calcaneus, tarsal and/or metatarsal bones.

### Growth Factors

In some embodiments, a growth factor and/or therapeutic agent may be disposed on or in the matrix by hand, electrospraying, ionization spraying or impregnating, vibratory dispersion (including sonication), nozzle spraying, compressed-air-assisted spraying, injecting, brushing and/or pouring. For example, a growth factor such as rhBMP-2 may be disposed on or in the biodegradable matrix by the surgeon before the biodegradable matrix is administered or the matrix may be pre-loaded with the growth factor by the manufacturer beforehand.

The biodegradable matrix may comprise at least one growth factor. These growth factors include osteoinductive agents (e.g., agents that cause new bone growth in an area where there was none) and/or osteoconductive agents (e.g., agents that cause ingrowth of cells into and/or through the matrix). Osteoinductive agents can be polypeptides or polynucleotides compositions. Polynucleotide compositions of the osteoinductive agents include, but are not limited to, isolated Bone Morphogenic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), Platelet derived growth factor, (PDGF or rhPDGF), Insulin-like growth factor (IGF) or Transforming Growth Factor beta (TGF-beta) polynucleotides. Polynucleotide compositions of the osteoinductive agents include, but are not limited to, gene therapy vectors harboring polynucleotides encoding the osteoinductive polypeptide of interest. Gene therapy methods often utilize a polynucleotide, which codes for the osteoinductive polypeptide operatively linked or associated to a promoter or any other genetic elements necessary for the expression of the osteoinductive polypeptide by the target tissue. Such gene therapy and delivery techniques are known in the art (see, for example, International Publication No. WO90/11092, the disclosure of which is herein incorporated by reference in its entirety). Suitable gene therapy vectors include, but are not limited to, gene therapy vectors that do not integrate into the host genome. Alternatively, suitable gene therapy vectors include, but are not limited to, gene therapy vectors that integrate into the host genome.

In some embodiments, the polynucleotide is delivered in plasmid formulations. Plasmid DNA or RNA formulations refer to polynucleotide sequences encoding osteoinductive polypeptides that are free from any delivery vehicle that acts to assist, promote or facilitate entry into the cell, including viral sequences, viral particles, liposome formulations, lipofectin, precipitating agents or the like. Optionally, gene therapy compositions can be delivered in liposome formulations and lipofectin formulations, which can be prepared by methods well known to those skilled in the art. General methods are described, for example, in U.S. Pat. Nos. 5,593,972, 5,589,466, and 5,580,859, the disclosures of which are herein incorporated by reference in their entireties.

Gene therapy vectors further comprise suitable adenoviral vectors including, but not limited to for example, those described in U.S. Pat. No. 5,652,224, which is herein incorporated by reference.

Polypeptide compositions of the isolated osteoinductive agents include, but are not limited to, isolated Bone Morphogenic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), Platelet derived growth factor, (PDGF or rhPDGF), Insulin-like growth factor (IGF) or Transforming Growth Factor beta (TGF-beta707) polypeptides. Polypeptide compositions of the osteoinductive agents include, but are not limited to, full length proteins, fragments or variants thereof.

Variants of the isolated osteoinductive agents include, but are not limited to, polypeptide variants that are designed to increase the duration of activity of the osteoinductive agent in vivo. Typically, variant osteoinductive agents include, but are not limited to, full length proteins or fragments thereof that are conjugated to polyethylene glycol (PEG) moieties to increase their half-life in vivo (also known as pegylation). Methods of pegylating polypeptides are well known in the art (See, e.g., U.S. Pat. No. 6,552,170 and European Pat. No. 0,401,384 as examples of methods of generating pegylated polypeptides). In some embodiments, the isolated osteoinductive agent(s) are provided as fusion proteins. In one embodiment, the osteoinductive agent(s) are available as fusion proteins with the Fc portion of human IgG. In another embodiment, the osteoinductive agent(s) are available as hetero- or homodimers or multimers. Examples of some fusion proteins include, but are not limited to, ligand fusions between mature osteoinductive polypeptides and the Fc portion of human Immunoglobulin G (IgG). Methods of making fusion proteins and constructs encoding the same are well known in the art.

Isolated osteoinductive agents that are included within a matrix are typically sterile. In a non-limiting method, sterility is readily accomplished for example by filtration through sterile filtration membranes (e.g., 0.2 micron membranes or filters). In one embodiment, the matrix includes osteoinductive agents comprising one or more members of the family of Bone Morphogenic Proteins (“BMPs”). BMPs are a class of proteins thought to have osteoinductive or growth-promoting activities on endogenous bone tissue, or function as pro-collagen precursors. Known members of the BMP family include, but are not limited to, BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18 as well as polynucleotides or polypeptides thereof, as well as mature polypeptides or polynucleotides encoding the same.

BMPs utilized as osteoinductive agents comprise one or more of BMP-1; BMP-2; BMP-3; BMP-4; BMP-5; BMP-6; BMP-7; BMP-8; BMP-9; BMP-10; BMP-11; BMP-12; BMP-13; BMP-15; BMP-16; BMP-17; or BMP-18; as well as any combination of one or more of these BMPs, including full length BMPs or fragments thereof, or combinations thereof, either as polypeptides or polynucleotides encoding the polypeptide fragments of all of the recited BMPs. The isolated BMP osteoinductive agents may be administered as polynucleotides, polypeptides, full length protein or combinations thereof.

In another embodiment, isolated osteoinductive agents that are loaded in the matrix include osteoclastogenesis inhibitors to inhibit bone resorption of the bone tissue surrounding the site of implantation by osteoclasts. Osteoclast and osteoclastogenesis inhibitors include, but are not limited to, osteoprotegerin polynucleotides or polypeptides, as well as mature osteoprotegerin proteins, polypeptides or polynucleotides encoding the same. Osteoprotegerin is a member of the TNF-receptor superfamily and is an osteoblast-secreted decoy receptor that functions as a negative regulator of bone resorption. This protein specifically binds to its ligand, osteoprotegerin ligand (TNFSF11/OPGL), both of which are key extracellular regulators of osteoclast development.

Osteoclastogenesis inhibitors that can be loaded in the matrix further include, but are not limited to, chemical compounds such as bisphosphonate, 5-lipoxygenase inhibitors such as those described in U.S. Pat. Nos. 5,534,524 and 6,455,541 (the contents of which are herein incorporated by reference in their entireties), heterocyclic compounds such as those described in U.S. Pat. No. 5,658,935 (herein incorporated by reference in its entirety), 2,4-dioxoimidazolidine and imidazolidine derivative compounds such as those described in U.S. Pat. Nos. 5,397,796 and 5,554,594 (the contents of which are herein incorporated by reference in their entireties), sulfonamide derivatives such as those described in U.S. Pat. No. 6,313,119 (herein incorporated by reference in its entirety), or acylguanidine compounds such as those described in U.S. Pat. No. 6,492,356 (herein incorporated by reference in its entirety).

In another embodiment, isolated osteoinductive agents that can be loaded in the matrix include one or more members of the family of Connective Tissue Growth Factors (“CTGFs”). CTGFs are a class of proteins thought to have growth-promoting activities on connective tissues. Known members of the CTGF family include, but are not limited to, CTGF-1, CTGF-2, CTGF-4 polynucleotides or polypeptides thereof, as well as mature proteins, polypeptides or polynucleotides encoding the same.

In another embodiment, isolated osteoinductive agents that can be loaded in the matrix include one or more members of the family of Vascular Endothelial Growth Factors (“VEGFs”). VEGFs are a class of proteins thought to have growth-promoting activities on vascular tissues. Known members of the VEGF family include, but are not limited to, VEGF-A, VEGF-B, VEGF-C, VEGF-D, VEGF-E or polynucleotides or polypeptides thereof, as well as mature VEGF-A, proteins, polypeptides or polynucleotides encoding the same.

In another embodiment, isolated osteoinductive agents that can be loaded in the matrix include one or more members of the family of Transforming Growth Factor-beta (“TGFbetas”). TGF-betas are a class of proteins thought to have growth-promoting activities on a range of tissues, including connective tissues. Known members of the TGF-beta family include, but are not limited to, TGF-beta-1, TGF-beta-2, TGF-beta-3, polynucleotides or polypeptides thereof, as well as mature protein, polypeptides or polynucleotides encoding the same.

In another embodiment, isolated osteoinductive agents that can be loaded in the matrix include one or more Growth Differentiation Factors (“GDFs”). Known GDFs include, but are not limited to, GDF-1, GDF-2, GDF-3, GDF-7, GDF-10, GDF-11, and GDF-15. For example, GDFs useful as isolated osteoinductive agents include, but are not limited to, the following GDFs: GDF-1 polynucleotides or polypeptides corresponding to GenBank Accession Numbers M62302, AAA58501, and AAB94786, as well as mature GDF-1 polypeptides or polynucleotides encoding the same. GDF-2 polynucleotides or polypeptides corresponding to GenBank Accession Numbers BC069643, BC074921, Q9UK05, AAH69643, or AAH74921, as well as mature GDF-2 polypeptides or polynucleotides encoding the same. GDF-3 polynucleotides or polypeptides corresponding to GenBank Accession Numbers AF263538, BC030959, AAF91389, AAQ89234, or Q9NR23, as well as mature GDF-3 polypeptides or polynucleotides encoding the same. GDF-7 polynucleotides or polypeptides corresponding to GenBank Accession Numbers AB158468, AF522369, AAP97720, or Q7Z4P5, as well as mature GDF-7 polypeptides or polynucleotides encoding the same. GDF-10 polynucleotides or polypeptides corresponding to GenBank Accession Numbers BC028237 or AAH28237, as well as mature GDF-10 polypeptides or polynucleotides encoding the same.

GDF-11 polynucleotides or polypeptides corresponding to GenBank Accession Numbers AF100907, NP_{—}005802 or 095390, as well as mature GDF-11 polypeptides or polynucleotides encoding the same. GDF-15 polynucleotides or polypeptides corresponding to GenBank Accession Numbers BC008962, BC000529, AAH00529, or NP_{—}004855, as well as mature GDF-15 polypeptides or polynucleotides encoding the same.

In another embodiment, isolated osteoinductive agents that can be loaded in the matrix include Cartilage Derived Morphogenic Protein (CDMP) and Lim Mineralization Protein (LMP) polynucleotides or polypeptides. Known CDMPs and LMPs include, but are not limited to, CDMP-1, CDMP-2, LMP-1, LMP-2, or LMP-3.

CDMPs and LMPs useful as isolated osteoinductive agents that can be loaded in the matrix include, but are not limited to, the following CDMPs and LMPs: CDMP-1 polynucleotides and polypeptides corresponding to GenBank Accession Numbers NM_{—}000557, U13660, NP_{—}000548 or P43026, as well as mature CDMP-1 polypeptides or polynucleotides encoding the same. CDMP-2 polypeptides corresponding to GenBank Accession Numbers or P55106, as well as mature CDMP-2 polypeptides. LMP-1 polynucleotides or polypeptides corresponding to GenBank Accession Numbers AF345904 or AAK30567, as well as mature LMP-1 polypeptides or polynucleotides encoding the same. LMP-2 polynucleotides or polypeptides corresponding to GenBank Accession Numbers AF345905 or AAK30568, as well as mature LMP-2 polypeptides or polynucleotides encoding the same. LMP-3 polynucleotides or polypeptides corresponding to GenBank Accession Numbers AF345906 or AAK30569, as well as mature LMP-3 polypeptides or polynucleotides encoding the same.

In another embodiment, isolated osteoinductive agents that can be loaded in the matrix include one or more members of any one of the families of Bone Morphogenic Proteins (BMPs), Connective Tissue Growth Factors (CTGFs), Vascular Endothelial Growth Factors (VEGFs), Osteoprotegerin or any of the other osteoclastogenesis inhibitors, Growth Differentiation Factors (GDFs), Cartilage Derived Morphogenic Proteins (CDMPs), Lim Mineralization Proteins (LMPs), or Transforming Growth Factor-betas (TGF-betas), as well as mixtures or combinations thereof.

In another embodiment, the one or more isolated osteoinductive agents that can be loaded in the matrix are selected from the group consisting of BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18, or any combination thereof; CTGF-1, CTGF-2, CGTF-3, CTGF-4, or any combination thereof; VEGF-A, VEGF-B, VEGF-C, VEGF-D, VEGF-E, or any combination thereof; GDF-1, GDF-2, GDF-3, GDF-7, GDF-10, GDF-11, GDF-15, or any combination thereof; CDMP-1, CDMP-2, LMP-1, LMP-2, LMP-3, and/or any combination thereof; Osteoprotegerin; TGF-beta-1, TGF-beta-2, TGF-beta-3, or any combination thereof; or any combination of one or more members of these groups.

In some embodiments, BMP-2, BMP-7 and/or GDF-5 may be used at 1-2 mg/cc of matrix. The concentrations of growth factor can be varied based on the desired length or degree of osteogenic effects desired. Similarly, one of skill in the art will understand that the duration of sustained release of the growth factor can be modified by the manipulation of the compositions of the matrix, such as for example, microencapsulation of the growth factor within polymers. The sustained release matrix can therefore be designed to provide customized time release of growth factors that stimulate the natural healing process.

The growth factor may contain inactive materials such as buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfate, sodium bisulfite, sodium thiosulfate, thimerosal, methylparaben, polyvinyl alcohol and phenylethyl alcohol; solubility adjusting agents; stabilizers; and/or cohesion modifiers. In some embodiments, the growth factor may comprise sterile and/or preservative free material.

These above inactive ingredients may have multi-functional purposes including the carrying, stabilizing and controlling the release of the growth factor and/or other therapeutic agent(s). The sustained release process, for example, may be by a solution-diffusion mechanism or it may be governed by an erosion-sustained process.

In some embodiments, a pharmaceutically acceptable formulation comprising a growth factor is provided, wherein the formulation is a freeze-dried or lyophilized formulation. Typically, in the freeze-dried or lyophilized formulation an effective amount of a growth factor is provided. Lyophilized formulations can be reconstituted into solutions, suspensions, emulsions, or any other suitable form for administration or use. The lyophilized formulation may comprise the liquid used to reconstitute the growth factor. Lyophilized formulations are typically first prepared as liquids, then frozen and lyophilized. The total liquid volume before lyophilization can be less, equal to, or more than the final reconstituted volume of the lyophilized formulation. The lyophilization process is well known to those of ordinary skill in the art, and typically includes sublimation of water from a frozen formulation under controlled conditions.

Lyophilized formulations can be stored at a wide range of temperatures. Lyophilized formulations may be stored at or below 30° C., for example, refrigerated at 4° C., or at room temperature (e.g., approximately 25° C.).

Lyophilized formulations of the growth factor are typically reconstituted for use by addition of an aqueous solution to dissolve the lyophilized formulation. A wide variety of aqueous solutions can be used to reconstitute a lyophilized formulation. In some embodiments, lyophilized formulations can be reconstituted with a solution containing water (e.g., USP WFI, or water for injection) or bacteriostatic water (e.g., USP WFI with 0.9% benzyl alcohol). However, solutions comprising buffers and/or excipients and/or one or more pharmaceutically acceptable carries can also be used. In some embodiments, the solutions do not contain any preservatives (e.g., are preservative free).

### Application of the Growth Factor to the Matrix

In some embodiments, a therapeutic agent (including one or more growth factors) may be disposed on or in the interior of the matrix by hand, electrospraying, ionization spraying or impregnating, vibratory dispersion (including sonication), nozzle spraying, compressed-air-assisted spraying, injecting, brushing and/or pouring.

Application of the growth factor to the matrix may occur at the time of surgery or by the manufacturer or in any other suitable manner. For example, the growth factor may be further reconstituted using a syringe and the syringe can be placed into the interior of the matrix via insertion of a needle or cannula (piercing the matrix membrane) and placing it into the interior of the matrix and injecting the growth factor so it is evenly distributed throughout the porous interior.

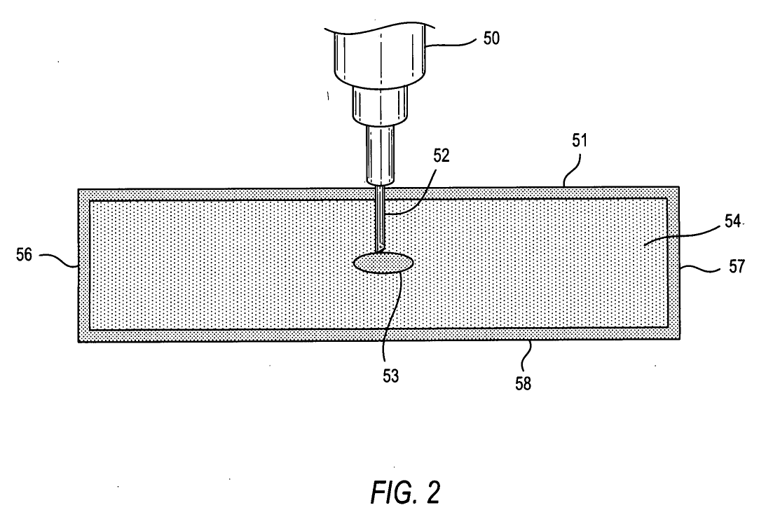

FIG. 2 is a magnified side sectional view of an embodiment of the matrix being filled with the growth factor. In this illustrated embodiment, the matrix is shown as a bi-layered sponge. The matrix is being loaded with a growth factor liquid 53 via syringe 50 that contains the growth factor and needle 52 that pierces the membrane 51 and loads the growth factor 53 within the porous interior 54 of the collagen sponge. Once injected, the porous interior of the collagen sponge will then contract in size as the growth factor is evenly distributed therein. The porous interior holds the growth factor within the matrix and because the interior is porous, the growth factor is evenly distributed throughout the matrix when growth factor is injected into the matrix.

The matrix has a less porous or less permeable membrane (shown as 51, 56, 57, and 58) disposed on the more porous interior 54. The membrane (shown as 51, 56, 57 and 58) will hold the growth factor within the interior of the matrix and as the membrane degrades, growth factor will be released into the environment surrounding the matrix (e.g., bone defect, osteochondral defect, etc.). In this way, the matrix provided serves to prolong residence time of the growth factor in or on the matrix by reducing the influx of blood and other bodily fluid into the matrix. This influx of blood and fluid, in some embodiments, can de-bind the growth factor and cause unwanted release of it. Thus, the matrix can maintain the efficacy of the growth factor over time to promote bone and/or cartilage growth at a target site. The membrane will also reduce or eliminate and initial burst effect where a bolus dose of the growth factor will be immediately released.

Although the matrix is shown as a collagen sponge, it will be understood by one of ordinary skill in the art that the matrix can be made of other material in different shapes and sizes depending on the condition being treated.

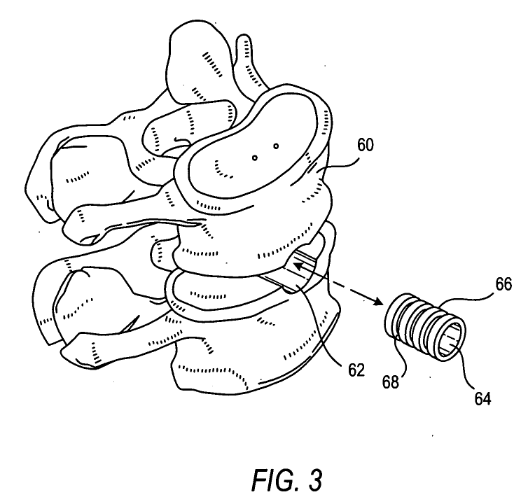

FIG. 3 illustrates a side view of a vertebrae and the implantable matrix shaped as a plug with ridges for implantation at a target tissue site (e.g., an intervertebral site). Shown in FIG. 3 is the matrix configured to be implanted at a bone defect 62 in the anterior side of the vertebral column. The implantable matrix has porous interior 64 containing the growth factor (e.g., BMP-2), which has a less porous membrane 68 disposed on the porous interior. The membrane 68 has ridges 66 disposed on the outside of the membrane so that the implantable matrix is retained at the bone defect 62. As the membrane degrades, growth factor will be locally released and cells and/or tissue are allowed to innervate the implantable matrix, which enhances bone growth.

In some embodiments, the growth factor may be applied to the matrix (i.e., collagen) prior to combining the materials and forming it into the final matrix shape. Indeed, the growth factor can be blended into the natural or synthetic polymer (i.e., POE) and poured into molds of the final shape of the matrix. Alternatively, the growth factor, such as a bone morphogenetic protein in a suitable liquid carrier, may be applied onto and/or into the porous loaded matrix after forming it into the final shape by soaking, dripping, injecting, spraying, etc.

In some embodiments, the interior of the matrix is loaded with BMP that functions as an osteoinductive factor. Indeed, the preferred osteoinductive factors are the recombinant human bone morphogenetic proteins (rhBMPs) because they are available in unlimited supply and do not transmit infectious diseases. In some embodiments, the bone morphogenetic protein is a rhBMP-2, rhBMP-4, rhBMP-7, or heterodimers thereof.

Recombinant BMP-2 can be used at a concentration of about 0.4 mg/ml to about 10.0 mg/ml, preferably near 1.5 mg/ml. However, any bone morphogenetic protein is contemplated including bone morphogenetic proteins designated as BMP-1 through BMP-18. BMPs are available from Wyeth, Cambridge, Mass. and the BMPs and genes encoding them may also be prepared by one skilled in the art as described in U.S. Pat. No. 5,187,076 to Wozney et al.; U.S. Pat. No. 5,366,875 to Wozney et al.; U.S. Pat. No. 4,877,864 to Wang et al.; U.S. Pat. No. 5,108,922 to Wang et al.; U.S. Pat. No. 5,116,738 to Wang et al.; U.S. Pat. No. 5,013,649 to Wang et al.; U.S. Pat. No. 5,106,748 to Wozney et al.; and PCT Patent Nos. WO93/00432 to Wozney et al.; WO94/26893 to Celeste et al.; and WO94/26892 to Celeste et al. All osteoinductive factors are contemplated whether obtained as above or isolated from bone. Methods for isolating bone morphogenetic protein from bone are described, for example, in U.S. Pat. No. 4,294,753 to Urist and Urist et al., 81 PNAS 371, 1984.

In some embodiments, the lyophilized growth factor (e.g., BMP) can be disposed in a vial by the manufacturer and then the surgeon can mix the diluent with the lyophilized growth factor. This mixture can then be injected into the porous interior of the matrix, as discussed above in FIG. 2. The matrix then can be parenterally administered to the target tissue site. The term “parenteral” as used herein refers to modes of administration which bypass the gastrointestinal tract, and include for example, intramuscular, intraperitoneal, intrasternal, subcutaneous, intra-operatively, intrathecally, intradiscally, peridiscally, epidurally, perispinally, intraarticular or combinations thereof.

The amount of growth factor, (e.g., bone morphogenic protein) may be sufficient to cause bone and/or cartilage growth. In some embodiments, the growth factor is rhBMP-2 and is contained in one or more matrices in an amount of from 1 to 2 mg per cubic centimeter of the biodegradable matrix. In some embodiments, the amount of rhBMP-2 morphogenic protein is from 2.0 to 2.5 mg per cubic centimeter (cc) of the biodegradable matrix.

In some embodiments, the growth factor is supplied in a liquid carrier (e.g., an aqueous buffered solution). Exemplary aqueous buffered solutions include, but are not limited to, TE, HEPES (2-[4-(2-hydroxyethyl)-1-piperazinyl]ethanesulfonic acid), MES (2-morpholinoethanesulfonic acid), sodium acetate buffer, sodium citrate buffer, sodium phosphate buffer, a Tris buffer (e.g., Tris-HCL), phosphate buffered saline (PBS), sodium phosphate, potassium phosphate, sodium chloride, potassium chloride, glycerol, calcium chloride or a combination thereof. In various embodiments, the buffer concentration can be from about 1 mM to 100 mM. In some embodiments, the BMP-2 is provided in a vehicle (including a buffer) containing sucrose, glycine, L-glutamic acid, sodium chloride, and/or polysorbate 80.

In some embodiments, upon implantation of the matrix or components that contact the matrix (e.g., plugs that are separate from the matrix on implantation), compression of the matrix is reduced or eliminated. As discussed above, if unwanted compression occurs, this causes the buffer from the bone growth factor to leak from the matrix, which causes higher concentrations of the growth factor (e.g., 2 mg to 2.5 mg of rhBMP-2 per cc of matrix) to remain on the matrix. This high concentration of growth factor may lead to local transient bone resorption and excess osteoclast formation and bone breakdown. This may result in poor integration of the matrix with surrounding host tissue and a failed repair. Thus, by employing a membrane disposed on the matrix, unwanted leakage is reduced or avoided. In some embodiments, localized release of the growth factor may cause local irritation to the surrounding tissue. In some embodiments, the leaking of growth factor from the matrix may reduce a stable microenvironment for new bone and/or cartilage growth. It also may cause the matrix to fail to retain its full efficacy over time to maximally promote bone growth at a target site.

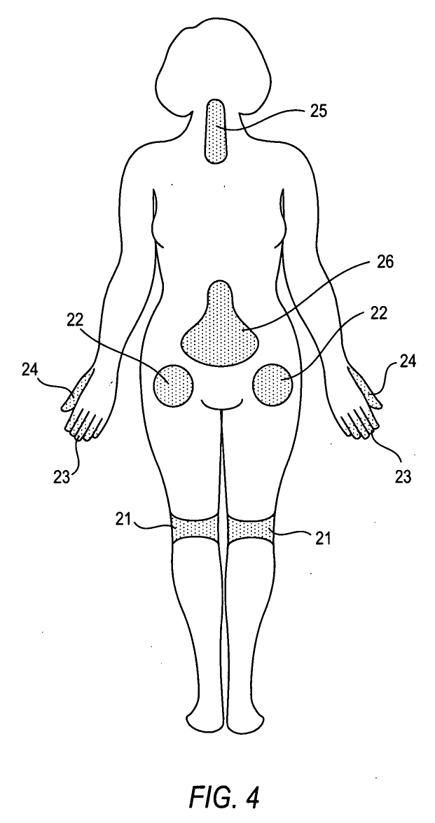

FIG. 4 illustrates a number of common locations within a patient that may be sites at which the matrix can be implanted before, during or after surgery. It will be recognized that the locations illustrated in FIG. 4 are merely exemplary of the many different locations within a patient that may be at which the matrix can be implanted. For example, the matrix may be implanted at a patient's knees 21, hips 22, fingers 23, thumbs 24, neck 25, and spine 26. Thus, during or following these surgeries, the matrix may be implanted at these or other target tissue sites (e.g., spinal disc space, spinal canal, soft tissue surrounding the spine, nerve root, bone muscle, etc).

### Additional Therapeutic Agents

The growth factors of the present application may be disposed on or in the matrix with other therapeutic agents. For example, the growth factor may be disposed on or in the carrier by electrospraying, ionization spraying or impregnating, vibratory dispersion (including sonication), nozzle spraying, compressed-air-assisted spraying, brushing and/or pouring.

Exemplary therapeutic agents include but are not limited to IL-1 inhibitors, such Kineret® (anakinra), which is a recombinant, non-glycosylated form of the human interleukin-1 receptor antagonist (IL-1Ra), or AMG 108, which is a monoclonal antibody that blocks the action of IL-1. Therapeutic agents also include excitatory amino acids such as glutamate and aspartate, antagonists or inhibitors of glutamate binding to NMDA receptors, AMPA receptors, and/or kainate receptors. Interleukin-1 receptor antagonists, thalidomide (a TNF-α release inhibitor), thalidomide analogues (which reduce TNF-α production by macrophages), quinapril (an inhibitor of angiotensin II, which upregulates TNF-α), interferons such as IL-11 (which modulate TNF-α receptor expression), and aurin-tricarboxylic acid (which inhibits TNF-α), may also be useful as therapeutic agents for reducing inflammation. It is further contemplated that where desirable a pegylated form of the above may be used. Examples of still other therapeutic agents include NF kappa B inhibitors such as antioxidants, such as dithiocarbamate, and other compounds, such as, for example, sulfasalazine.

Examples of therapeutic agents suitable for use also include, but are not limited to, an anti-inflammatory agent, analgesic agent, or osteoinductive growth factor or a combination thereof. Anti-inflammatory agents include, but are not limited to, apazone, celecoxib, diclofenac, diflunisal, enolic acids (piroxicam, meloxicam), etodolac, fenamates (mefenamic acid, meclofenamic acid), gold, ibuprofen, indomethacin, ketoprofen, ketorolac, nabumetone, naproxen, nimesulide, salicylates, sulfasalazine[2-hydroxy-5-[-4-[C2-pyridinylamino)sulfonyl]azo]benzoic acid, sulindac, tepoxalin, and tolmetin; as well as antioxidants, such as dithiocarbamate, steroids, such as cortisol, cortisone, hydrocortisone, fludrocortisone, prednisone, prednisolone, methylprednisolone, triamcinolone, betamethasone, dexamethasone, beclomethasone, fluticasone or a combination thereof.

Suitable analgesic agents include, but are not limited to, acetaminophen, bupivicaine, fluocinolone, lidocaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, mepiridine, methadone, morphine, nalbuphine, opium, oxycodone, papaveretum, pentazocine, pethidine, phenoperidine, piritramide, dextropropoxyphene, remifentanil, tilidine, tramadol, codeine, dihydrocodeine, meptazinol, dezocine, eptazocine, flupirtine, amitriptyline, carbamazepine, gabapentin, pregabalin, or a combination thereof. In some embodiments, a statin may be used. Statins include, but is not limited to, atorvastatin, simvastatin, pravastatin, cerivastatin, mevastatin (see U.S. Pat. No. 3,883,140, the entire disclosure is herein incorporated by reference), velostatin (also called synvinolin; see U.S. Pat. Nos. 4,448,784 and 4,450,171 these entire disclosures are herein incorporated by reference), fluvastatin, lovastatin, rosuvastatin and fluindostatin (Sandoz XU-62-320), dalvastain (EP Appln. Publn. No. 738510 A2, the entire disclosure is herein incorporated by reference), eptastatin, pitavastatin, or pharmaceutically acceptable salts thereof or a combination thereof. In various embodiments, the statin may comprise mixtures of (+)R and (−)-S enantiomers of the statin. In various embodiments, the statin may comprise a 1:1 racemic mixture of the statin.

### Kits

The matrix, growth factor and devices to administer the implantable matrix composition may be sterilizable. In various embodiments, one or more components of the matrix, and/or medical device to administer it may be sterilizable by radiation in a terminal sterilization step in the final packaging. Terminal sterilization of a product provides greater assurance of sterility than from processes such as an aseptic process, which require individual product components to be sterilized separately and the final package assembled in a sterile environment.

Typically, in various embodiments, gamma radiation is used in the terminal sterilization step, which involves utilizing ionizing energy from gamma rays that penetrates deeply in the device. Gamma rays are highly effective in killing microorganisms, they leave no residues nor have sufficient energy to impart radioactivity to the device. Gamma rays can be employed when the device is in the package and gamma sterilization does not require high pressures or vacuum conditions, thus, package seals and other components are not stressed. In addition, gamma radiation eliminates the need for permeable packaging materials.

In some embodiments, the implantable matrix may be packaged in a moisture resistant package and then terminally sterilized by gamma irradiation. In use the surgeon removes the one or all components from the sterile package for use.

In various embodiments, electron beam (e-beam) radiation may be used to sterilize one or more components of the matrix. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity.

Other methods may also be used to sterilize the implantable matrix and/or one or more components of the matrix, including, but not limited to, gas sterilization, such as, for example, with ethylene oxide or steam sterilization.

In various embodiments, a kit is provided comprising the growth factor, matrix, and/or diluents. The kit may include additional parts along with the implantable matrix combined together to be used to implant the matrix (e.g., wipes, needles, syringes, etc.). The kit may include the matrix in a first compartment. The second compartment may include a vial holding the growth factor, diluent and any other instruments needed for the localized drug delivery. A third compartment may include gloves, drapes, wound dressings and other procedural supplies for maintaining sterility of the implanting process, as well as an instruction booklet, which may include a chart that shows how to implant the matrix after reconstituting the growth factor. A fourth compartment may include additional needles and/or sutures. Each tool may be separately packaged in a plastic pouch that is radiation sterilized. A fifth compartment may include an agent for radiographic imaging. A cover of the kit may include illustrations of the implanting procedure and a clear plastic cover may be placed over the compartments to maintain sterility.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. An implantable matrix configured to fit at or near a target tissue site, the matrix comprising: a porous interior configured to release a growth factor and to allow influx of at least progenitor, bone and/or cartilage cells therein; and a biodegradable membrane disposed on the porous interior, the biodegradable membrane being less porous than the porous interior and configured to retain the growth factor and release the growth factor from the porous interior as the biodegradable membrane degrades at or near the target tissue site.

2. An implantable matrix according to claim 1, wherein (i) the porous interior and/or biodegradable membrane comprises at least one of collagen, a resorbable polymer, gelatin, a resorbable ceramic, hyaluronic acid, chitosan or combinations thereof or (ii) the biodegradable membrane allows at least progenitor, bone and/or cartilage cells into the matrix and growth factor out of the matrix as the membrane degrades.

3. An implantable matrix according to claim 1, wherein the growth factor is distributed evenly throughout the porous interior of the matrix by injecting the growth factor through the biodegradable membrane using a needle or cannula.

4. An implantable matrix according to claim 1, wherein the porous interior of the matrix comprises a plurality of pores and the biodegradable membrane is impregnated or coated on the porous interior of the matrix to clog the plurality of pores to reduce their porosity.

5. An implantable matrix according to claim 1, wherein the matrix is in the form of a bi-layered sponge, plug, pin, peg, or cylinder and further comprises autograft, allograft or xenograft progenitor, bone and/or cartilage cells seeded within the matrix.

6. An implantable matrix according to claim 1, wherein the growth factor comprises bone morphogenic protein-2 solution; and the porous interior and biodegradable membrane comprise collagen.

7. An implantable matrix according to claim 1, wherein the biodegradable membrane degrades over a period of at least 3 days to release growth factor and allow progenitor, bone and/or cartilage cells into the matrix.

8. An implantable matrix according to claim 1, wherein the matrix is biodegradable and holds the growth factor when the matrix is in an uncompressed state.

9. An implantable matrix according to claim 1, wherein the porous interior matrix comprises a plurality of pores having a pore size above 10 μm and the membrane comprises no initial pores or a plurality of pores having a pore size below 100 μm.

10. An implantable matrix configured to fit at or near a target tissue site, the matrix comprising: a porous biodegradable interior configured to release a growth factor and to allow influx of at least progenitor, bone and/or cartilage cells therein; and a biodegradable membrane disposed on the porous biodegradable interior, the biodegradable membrane being less porous than the biodegradable interior and configured to retain the growth factor and release the growth factor from the porous biodegradable interior as the biodegradable membrane degrades at or near the target tissue site.

11. An implantable matrix according to claim 10, wherein the porous biodegradable interior and/or biodegradable membrane comprises at least one of collagen, a resorbable polymer, gelatin, a resorbable ceramic, hyaluronic acid, chitosan or combinations thereof.

12. An implantable matrix according to claim 10, wherein the growth factor is distributed evenly throughout the porous biodegradable interior of the matrix.

13. An implantable matrix according to claim 10, wherein the porous biodegradable interior of the matrix comprises a plurality of pores and the biodegradable membrane is impregnated or coated on the porous interior of the matrix to clog the plurality of pores to reduce their porosity.

14. An implantable matrix according to claim 10, wherein the matrix comprises a bi-layered matrix having the porous biodegradable interior in one layer and the biodegradable membrane disposed in one layer on the porous biodegradable interior layer.

15. An implantable matrix according to claim 10, wherein the growth factor comprises bone morphogenic protein-2 in a buffered solution; and the porous biodegradable interior and the biodegradable membrane both comprise collagen.

16. An implantable matrix according to claim 10, wherein the biodegradable membrane degrades faster than the porous biodegradable interior of the matrix to release growth factor from the porous biodegradable interior and allow progenitor, bone and/or cartilage cells into the matrix.

17. An implantable matrix according to claim 10, wherein the matrix comprises a biodegradable polymer and holds the growth factor when the matrix is in an uncompressed state.

18. A method for making an implantable collagen matrix, the method comprising: providing a porous collagen layer configured to release a growth factor and to allow influx of at least progenitor, bone and/or cartilage cells therein, and disposing a collagen membrane on the porous collagen layer, the collagen membrane being less porous than the porous collagen layer and configured to retain the growth factor.

19. A method for making an implantable collagen matrix according to claim 18, wherein the porous collagen layer comprises a plurality of pores having an average pore size above 50 μm and the collagen membrane comprises no pores or a plurality of pores having an average pore size below 50 μm and the collagen membrane is disposed on the collagen layer by heating, melting, or chemically treating a surface of the collagen layer to reduce the pore size of the porous collagen layer to form the collagen membrane.

20. A method for making an implantable collagen matrix according to claim 18, wherein the collagen membrane is disposed on the porous collagen layer by spraying collagen on a surface of the porous collagen layer to reduce the pore size of the porous collagen layer.
